(19) United States
(12) Patent Application Publication
(10) Pub. No.: US 2004/0111072 A1
(43) Pub. Date: Jun. 10, 2004

(54) Display arrangement for ostomy care

(76) Inventor: Megan Anne McKissick, Pittsburgh, PA (US)

Correspondence Address: DR. EDMOND S. MIKSCH, 3825 GREEN VALLEY DRIVE, PITTSBURGH, PA, 15235, US

(21) Appl. No.: 10/315,595

(22) Filed: Dec. 10, 2002

(51) Int. Cl. A61F 5/44

(52) U.S. Cl. 604/332

(57) ABSTRACT

A display arrangement for organizing and presenting supplies and implements needed for ostomy care has at least one display surface for the supplies and implements, the display surface having alphamerically sequenced indicia disposed thereon. The indicia include at least one indicator for at least one skin preparation product, at least one indicator for stoma measurement, at least one indicator for at least one wafer cutting implement, at least one indicator for a wafer, at least one indicator for at least one adhesive product; and at least one indicator for at a pouch.

### FIELD OF THE INVENTION

The present invention relates, in general, to ostomy care, and, more particularly, the instant invention relates to a system for displaying the instruments and supplies required for ostomy care.

### BACKGROUND OF THE INVENTION

Patients who have lost the normal function of the bladder or bowel due to birth defects, disease, or injury may require surgery which connects a portion of the bowel or urethra to an artificial opening (stoma) through which wastes are expelled. The opening is generally located in the abdomen. Such patients must wear a bag or pouch to receive the wastes. The bag or pouch must frequently be changed or drained, and the skin surrounding the stoma may require care. A number of instruments and supplies are needed for changing the bag or pouch, and it is very important for the patient to have all the instruments and supplies ready before starting the procedure.

Typically, the ostomy bag or pouch has a detachable connection to a wafer that is attached to the skin surrounding the stoma by an adhesive product such as paste. The wafer has an opening surrounded by a plastic ring having an annular ridge. The bag or pouch has an opening surrounded by a plastic ring having an annular groove. Connection is made by pressing the plastic rings together so the annular ridge of the ring on the wafer snaps into the annular groove on the bag or pouch.

When the bag or pouch is removed, it may also be necessary to replace the wafer. After the old wafer is detached from the patient's skin, the old adhesive product must be removed from the skin. This is done with a wipe having a solvent that is specific for the adhesive product that was used. The solvent may be oily. A towelette, which contains some moisture is used to remove the solvent. The skin surrounding the stoma is then prepared for the glue or paste by an adhesive prep. According to the present art, the adhesive prep may be a wipe or a spray. The adhesive prep leaves a sticky surface in the manner of contact cement.

A new wafer must be cut with a hole the correct size to fit around the stoma. A measurement guide having circular cutouts is employed to measure the stoma, and the selected cutout is used as a template for drawing a circle on the wafer as a guide for cutting the hole in the wafer.

It is important for all of the implements and supplies needed for changing the bag or pouch to be available before the procedure is started. For the patient to return to normal life after surgery, it is necessary for the patient, or other untrained care givers, to perform the ostomy care procedures. The process may be quite daunting to the patient or an unskilled caregiver who is confronted only with the supplies and implements in a disorganized manner.

Background art for surgery and dentistry includes a number of patents describing systems for displaying and holding implements for assisting surgeons or dentists in performing surgical or dental procedures.

U.S. Pat. No. 4,298,074: “Root Canal Equipment Packaging” describes a root canal treatment kit having trays with recesses for implements and materials employed for performing root canals.

U.S. Pat. No. 4,266,669: “Anesthesiologist's Instrument Tray” teaches a disposable tray for anesthesiologists. The tray has a central compartment for the patient's head, and recesses for sequenced instruments and medications on opposite sides of the patient's head.

U.S. Pat. No. 5,525,314: “Surgical Tool Container System” teaches a surgical tool container system for the organization, sterilization, storage and presentation of surgical instruments. The system has a tray with holes having elastic grommets for holding the shanks of the instruments.

U.S. Pat. No. 5,981,303: “Compact, Pocketed Wrapping for a Medical Procedure Kit and Process for Using Same” describes a medical procedure kit that is unrolled to unwrap components needed for the procedure in order of use.

U.S. Pat. No. 6,158,437: “Method of Performing a Surgical Procedure and Associated Surgical Instrument Support Tray” describes a method of performing a surgical procedure that includes providing an instrument supporting tray having a plurality of surgical instruments positioned adjacent instrument-identifying indicia disposed in predetermined relative positions, as preferred by a particular surgeon..

None of the background patents cited above were intended for use by the patient or by an unskilled caregiver, and none relate to ostomy care. The teachings of the patents cited above are nevertheless hereby incorporated into the present application by reference thereto.

### SUMMARY OF THE INVENTION

In one aspect, the present invention is a system for organizing and presenting supplies and implements needed for ostomy care. It includes at least one display surface for the supplies and implements needed for ostomy care, the display surface including alphamerically sequenced indicia. The indicia include at least one indicator for skin preparation product(s), at least one indicator for stoma measurement, at least one indicator for wafer cutting implement(s), at least one indicator for a wafer, at least one indicator for adhesive product(s) and at least one indicator for a pouch or bag.

In an additional aspect, the present invention is an ostomy care system including implements and supplies for ostomy care. The system includes at least one skin preparation product, at least one device for stoma measurement, at least one wafer cutting implement, at least one wafer, at least one adhesive product, at least one pouch or bag and at least one display surface for the supplies and implements. The display surface includes alphamerically sequenced indicia. The indicia include at least one indicator for the skin preparation product(s), at least one indicator for stoma measurement implement(s), at least one indicator for wafer cutting implement(s), at least one indicator for a wafer, at least one indicator for adhesive product(s) and at least one indicator for a pouch or bag.

### OBJECTS OF THE INVENTION

It is therefore one of the primary objects of the present invention to provide a checklist for the implements and supplies needed for the care of a colostomy, ileostomy, or urostomy.

Another object of the present invention is to provide a training aid to assist a patient, unskilled caregiver, or skilled caregiver in ostomy care.

Still another object of the present invention is to enable a patient or caregiver to set up a sequenced display of the supplies and implements needed to change an ostomy bag and care for the skin surounding the stoma.

Yet another object of the present invention is to enable a patient or care giver to verify that all the materials and implements are available for changing an ostomy bag or pouch before the old bag or pouch is removed.

A further object of the present invention is to provide an ostomy care system that may be given to an ostomy patient on discharge from a hospital, the system including all implements and materials needed for ostomy care, as well as a board or tray for displaying them and for making them readily available.

It is an additional object of the present invention to provide an ostomy care display surface that is disposable.

Still yet another object of the present invention is to provide a display board for the supplies and implements needed for ostomy care that is easy to clean.

Another object of the present invention is to provide a display board for the supplies and implements needed for ostomy care that can be folded for storage or travel.

A still further object of the present invention is to provide a display board for the supplies and implements needed for ostomy care that can bridge across a sink.

An additional object of the present invention is to provide a display arrangement for ostomy care that a patient can use in their own home, or when travelling.

A further object of the present invention is to provide an ostomy care display surface having elevated or depressed areas to prevent ostomy care supplies and implements on the display surface from sliding.

Yet another object of the present invention is to provide a shopping aid to assist an ostomy patient or care giver in replenishing ostomy care supplies.

In addition to the various objects and advantages of the present invention which have been generally described above, there will be various other objects and advantages of the invention that will become more readily apparent to those persons who are skilled in the relevant art from the following more detailed description of the invention, particularly, when the detailed description is taken in conjunction with the attached drawing figures and with the appended claims.

### BRIEF DESCRIPTION OF THE DRAWINGS

FIG. 1 is a schematic illustration of the basic elements of ostomy collection systems of the prior art that are used for colostomies, ileostomies and urostomies.

FIG. 2 is a schematic illustration of skin preparation products and adhesive products of the prior art that are used for ostomy care.

FIG. 3 is a schematic illustration of implements of the prior art that are used for stoma measurement and wafer cutting.

FIG. 4 is a schematic illustration of a first display surface having indicia for ostomy care supplies and implements, according to the present invention.

FIG. 5 is a schematic illustration of a second display surface having indicia for ostomy care supplies and implements, according to the present invention.

FIG. 6 is a schematic illustration of a foldable pair of display surfaces for ostomy care supplies and implements, the surfaces connected by a vertical hinge.

FIG. 7 is a schematic illustration of a trifold of display surfaces for ostomy care supplies and implements, the surfaces connected by two vertical hinges.

FIG. 8 is a schematic illustration of a foldable pair of display surfaces for ostomy care supplies and implements, the surfaces connected by a hinge running from side to side.

FIG. 9 is a schematic illustration of a tray having a display surface having elevated portions and indentations to prevent sliding or rolling of ostomy care supplies and implements.

FIG. 10 is a schematic illustration of a paper or cardboard sheet having indicia for ostomy care supplies and implements.

FIG. 11 is a schematic illustration of alternative indicia for adhesive prep products.

FIG. 12 is a schematic illustration of alternative indicia for collection pouches for a patient having both a colostomy or ileostomy and also a urostomy.

FIG. 13 is a schematic illustration of an ostomy care system including a kit containing ostomy care supplies and implements, and a display surface for the supplies and implements.

### BRIEF DESCRIPTION OF THE PRESENTLY PREFERRED AND VARIOUS ALTERNATIVE EMBODIMENTS OF THE INVENTION

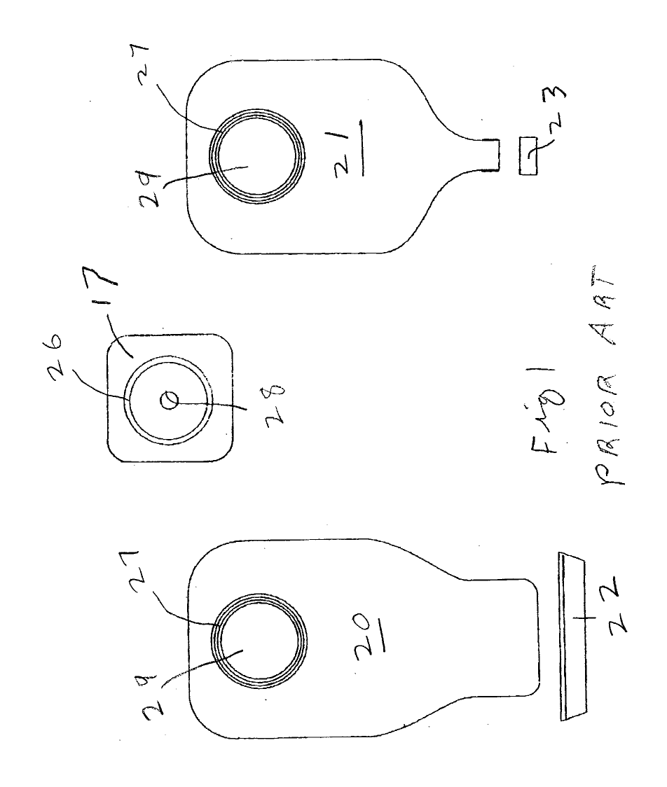

Attention is directed to FIGS. 1 through 3 that illustrate supplies and implements used in the prior art for care of colostomies, ileostomies, and urostomies. FIG. 1 shows a collection pouch 20 (sometimes called a bag) that is used for colostomies and ileostomies, a pouch 21 used for urostomies and a wafer 17 which is employed with both pouch 20 and pouch 21. Wafer 17 is for attachment to the patient's skin around the stoma (not shown). Wafer 17 has a hole 28 for fitting around the stoma and has an annular ridge 26 that snaps into annular groove 27 in pouch 20 or pouch 21. Pouch 20 or pouch 21 has a hole 29 for receiving wastes from the stoma. Pouch 20 may be closed by a clip 22. Pouch 21 may be closed by a cap 23, or alternatively, a plug (not shown) or a spout (not shown).

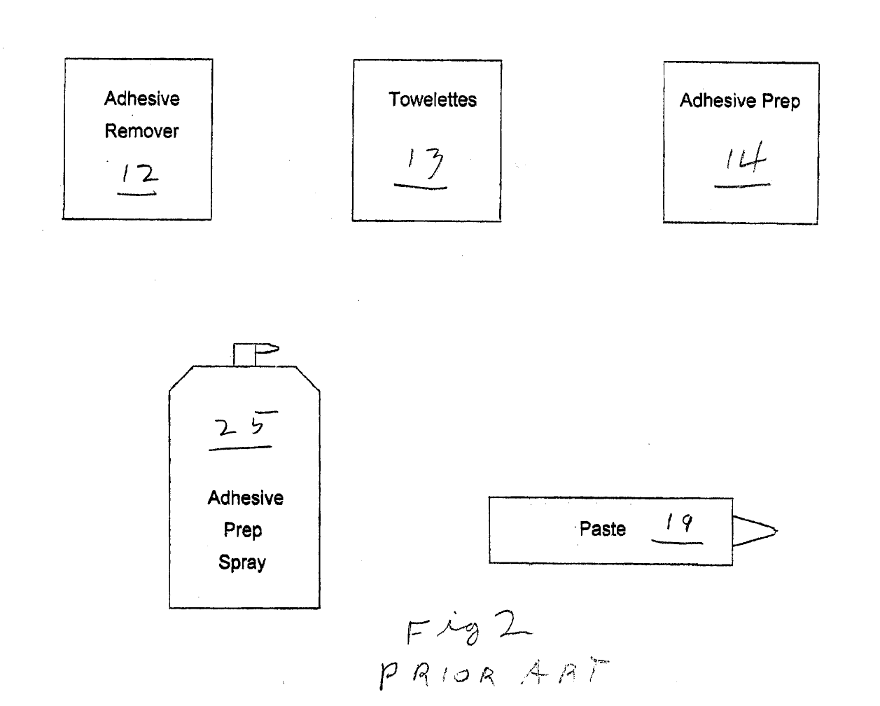

FIG. 2 illustrates skin preparation materials 12, 13,14 and 25, and paste 19 which are employed according to the prior art. When the entire ostomy collection system needs to be replaced, the wafer of that system must be detached from the skin. After the wafer is removed, some residual adhesive remains on the skin. The adhesive remover 12 is employed to remove that residual adhesive. Adhesive remover 12 is, generally, a wipe that contains a solvent that is specific for the adhesive product which was used. It, in turn, leaves an oily residue on the skin. A moist wipe 13, referred to as a towelette, is used to remove the oily residue. The towelette, generally, is hypoallergenic, and alcohol free. It may contain aloe, and may be scented.

An adhesive prep 14 or 25 is used to prepare the skin for adhesion of a new wafer. It places a sticky layer on the skin, which is similar to contact cement. Adhesive prep 14 is a wipe, which may be employed, or alternatively, adhesive prep spray 25 may be employed. Paste 19 is used to stick the wafer 17 to the patient's skin.

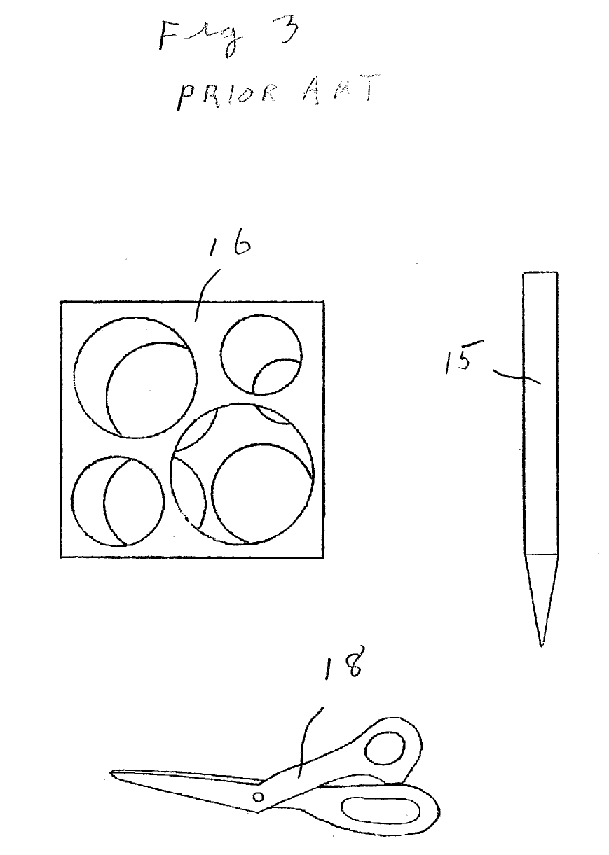

FIG. 3 illustrates implements used to measure the stoma and cut the wafer 17 to enlarge hole 28 so it fits around the stoma. The measurement guide 16 (shown folded as it is usually supplied) has circular holes of a number of different diameters. The smallest one that fits around the stoma is selected, and it is traced onto wafer 17 for cutting. Pen or pencil 15 is employed to mark wafer 17 for cutting. Scissors 18 may be employed for cutting wafer 17.

It is easily appreciated that the process of changing an ostomy pouch is complex, and it is a process that may need to be performed by the patient or by an unskilled caregiver. It is very important that all of the supplies and implements needed for the process be at hand before the process is commenced.

The purpose of the present invention is to provide an aid for organizing and presenting the various supplies and implements, and to indicate a sequence for their use.

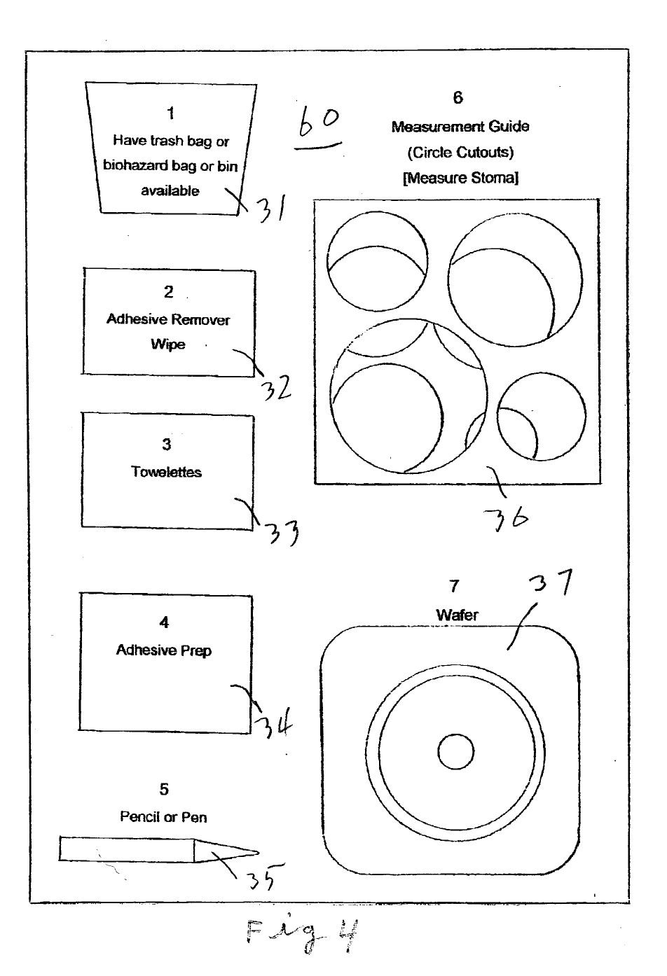

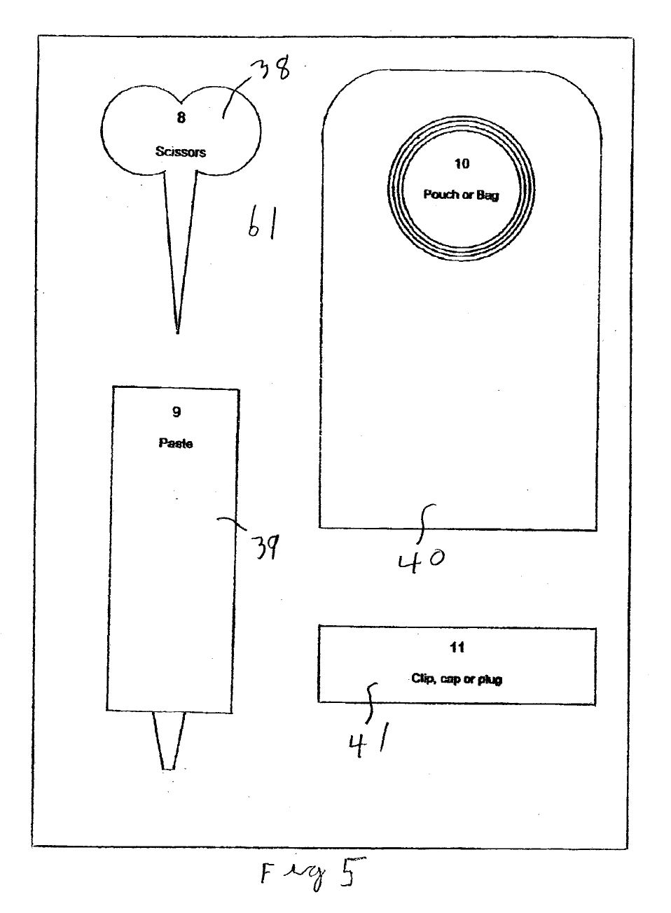

FIGS. 4 and 5 show display surfaces 60 and 61 having alphamerically sequenced indicia for the required supplies and implements. Indicator 31 is a reminder that a trash bag or biohazard bag or bin should be available before the process is begun. Indicator 32 provides a reminder that an adhesive remover wipe should be available and indicator 33 provides a reminder that a moist wipe or towelette should be available. Indicator 34 is for an adhesive prep, which may be either an adhesive prep wipe 14 or adhesive prep spray 25. The alphamerics 2, 3, 4, etc. on indicia 32, 33, 34, etc. remind a user of the sequence in which these products are to be employed.

In this figure, the indicia are sequenced by numerals. Alphabetic symbols could, alternatively, be employed. These could be in any language familiar to the intended user. The text for the various items could also be in a different language, familiar to the intended user.

Indicator 35 provides a reminder that a pen or pencil will be needed and indicator 36 is a reminder for a measurement guide 16 (shown in FIG. 3). Indicator 37 is a reminder for wafer 17. The patient or caregiver uses the circular apertures in measurement guide 16 to find the circular aperture that best fits around the stoma. The selected circular aperture is then traced onto wafer 17.

FIG. 5 is an illustration of a second display surface 61 which has additional indicia. Indicator 38 is a reminder that scissors 18 should be available. Scissors 18 are then used to enlarge hole 28 in wafer 17, following the tracing of the circular aperture in measurement guide 16 that was drawn on wafer 17.

Indicator 39 is a reminder that an adhesive product 19 should be available for attaching wafer 17 to the patient's skin. Indicator 40 is a reminder that a pouch 20 for a colostomy or ileostomy, or a pouch 21 for a urostomy should be available. Indicator 11 is an indicator for a clip 22, or a cap 23 or plug (not shown).

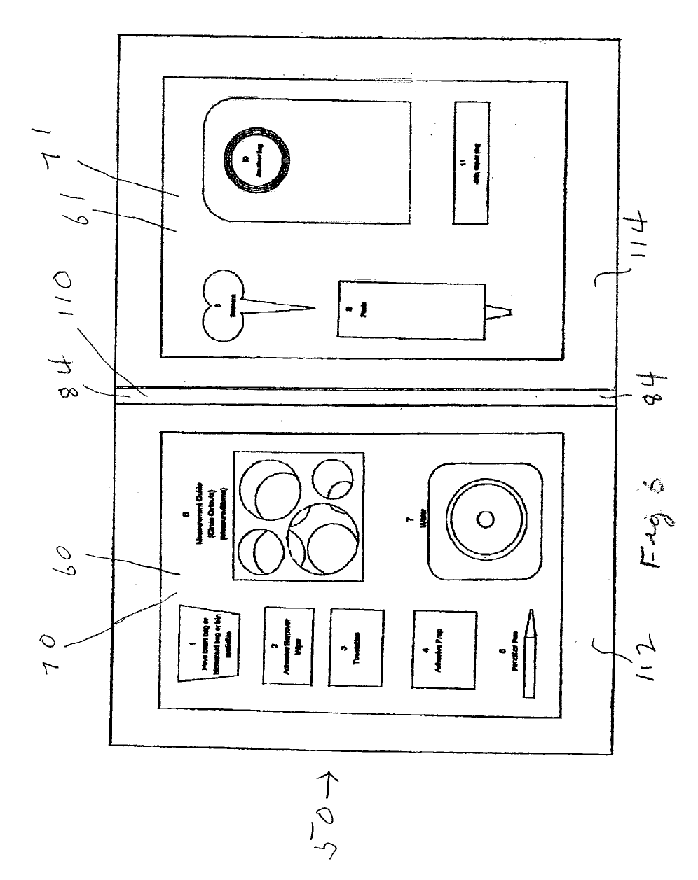

FIG. 6 shows a preferred physical embodiment that incorporates display surfaces 60 and 61 into an integrated display arrangement 50. Display surface 60 includes a paper or cardboard sheet 70 having indicia 31-37 printed thereon. Display surface 60 includes a covering transparent layer which, preferably, is acetate sheet 112. Display surface 61 includes a paper or cardboard sheet 71 having indicia 38-41 printed thereon. Indicia 31-41 are best seen in FIGS. 4-5. Display surface 61 includes a covering transparent layer which, preferable, is acetate sheet 114. To form the display arrangement 50, display surfaces 60 and 61 are laid over a larger acetate sheet 110 that is the full size of the arrangement 50. Acetate sheets 112 and 114 are then sealed to acetate sheet 110.

In FIG. 6, acetate sheet 110 is visible only in the center of display 50, between display surface 60 and display surface 61. Since there is only a single layer of acetate in this center region, it is flexible and acts as a hinge 84 running from the top to bottom of display arrangement 50. Display arrangement 50 may be folded for carrying or storage, and has a smooth, glossy surface that facilitates cleaning.

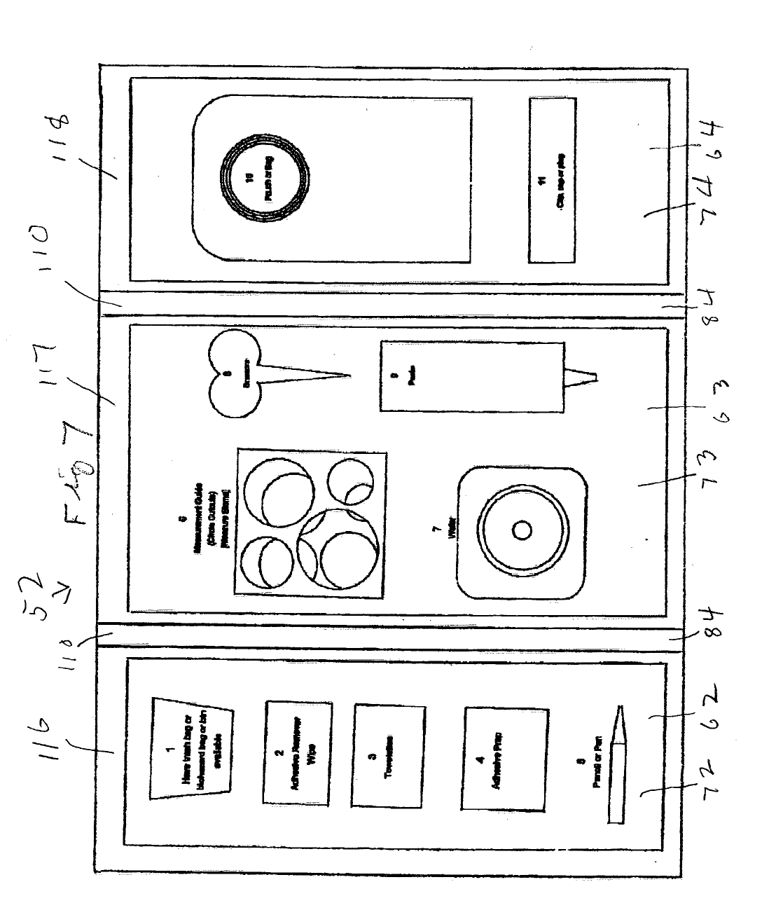

FIG. 7 illustrates another embodiment of the present invention. It is a trifold having display surfaces 62, 63 and 64. Display surface 62 comprises paper or cardboard sheet 72 having indicia 31-35 printed thereon. Display surface 63 comprises paper or cardboard sheet 73 having indicia 36-39 printed thereon. Display surface 64 comprises paper or cardboard sheet 74 having indicia 40-41 printed thereon. Indicia 31-41 are best seen in FIGS. 4 and 5.

Preferably, display surface 62 includes a covering acetate sheet 116, display surface 63 includes a covering acetate sheet 117 and display surface 64 includes a covering acetate sheet 118. Display surfaces 62, 63 and 64 are placed over a larger acetate sheet 110 that is the full size of display arrangement 52 and sheets 116,117 and 118 are sealed to larger acetate sheet 110.

A gap is left between display surface 62 and display surface 63 so there is only one layer of acetate in this gap (sheet 110). This forms a hinge 84 running from top to bottom of display arrangement 52. Likewise, a gap is left between display surface 63 and display surface 64 so there is only one layer of acetate in this gap (sheet 110). This also forms a hinge 84 running from top to bottom. Hence, display arrangement 52 may be folded into a trifold for travel or storage.

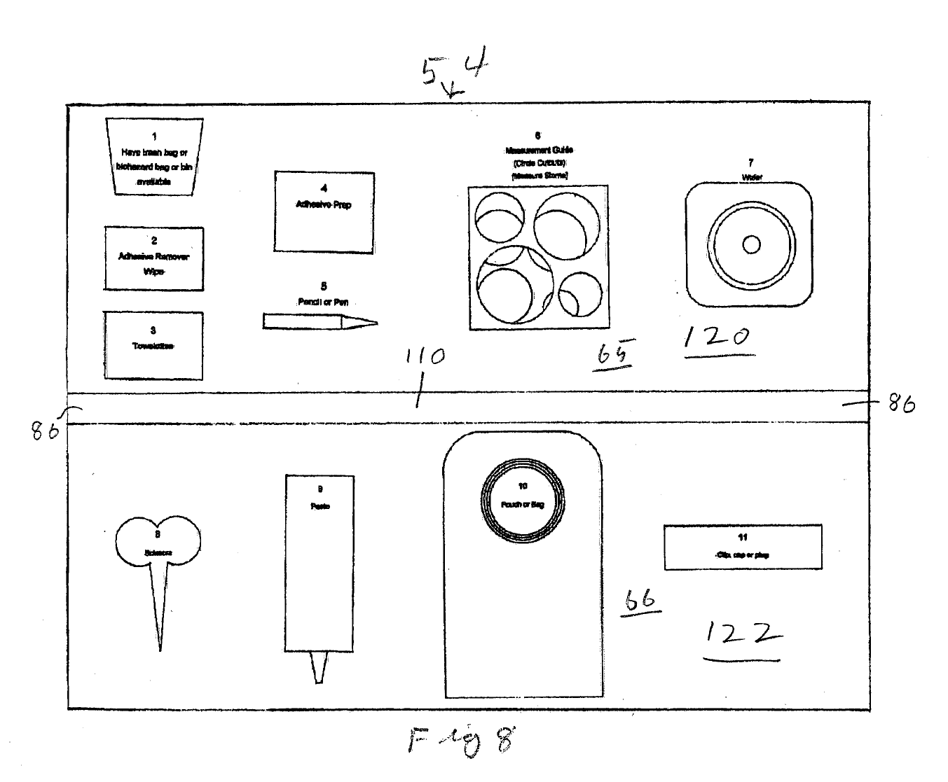

FIG. 8 illustrates a display arrangement 54, according to the present invention, having a hinge 86 running from the left side of the display arrangement 54 to the right side of display arrangement 54. Display arrangement 54 has an upper display surface 65 including acetate covering sheet 120 and it has a lower display surface 66 including acetate covering sheet 122.

These are laid over a larger acetate sheet 110 that is the full size of display arrangement 54. A gap is left between display surface 65 and display surface 66 to form a hinge 86 running from the left side to the right side of display arrangement 54. This arrangement may be folded for carrying or storage, and is designed to bridge across a sink where it may be placed for use.

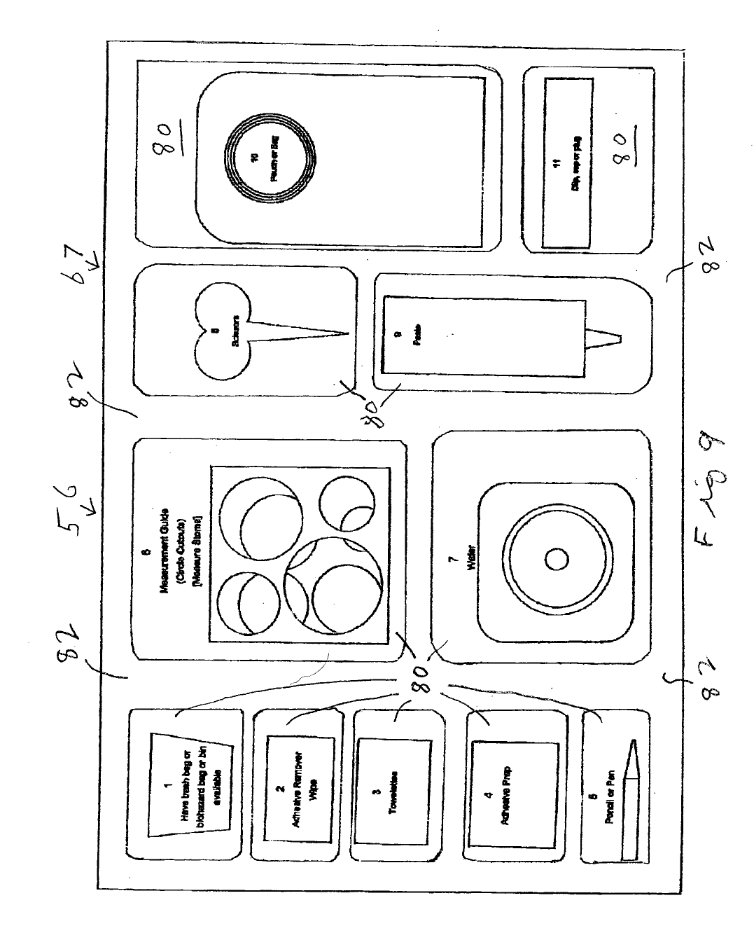

FIG. 9 illustrates a display arrangement 56 formed as a tray 67 having indentations 80 and elevated portions 82 so that the supplies and implements do not slide or roll. This configuration is particularly appropriate for use in a train or airplane.

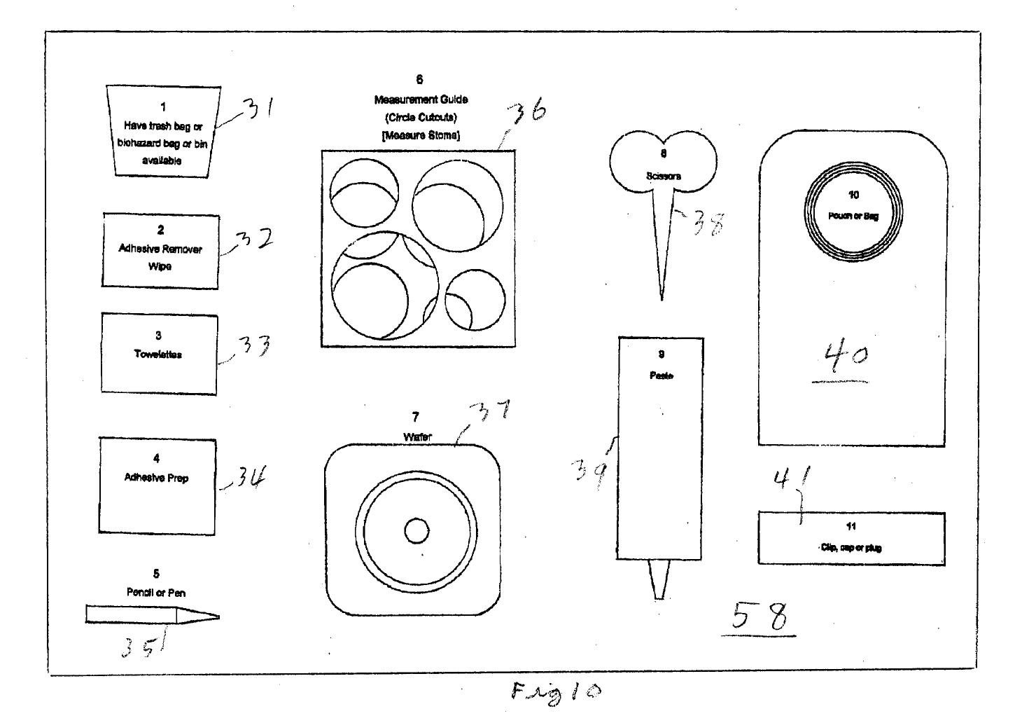

FIG. 10 is a disposable version of the invention. It comprises a sheet of paper or cardboard 58 having indicia 31-41 printed thereon.

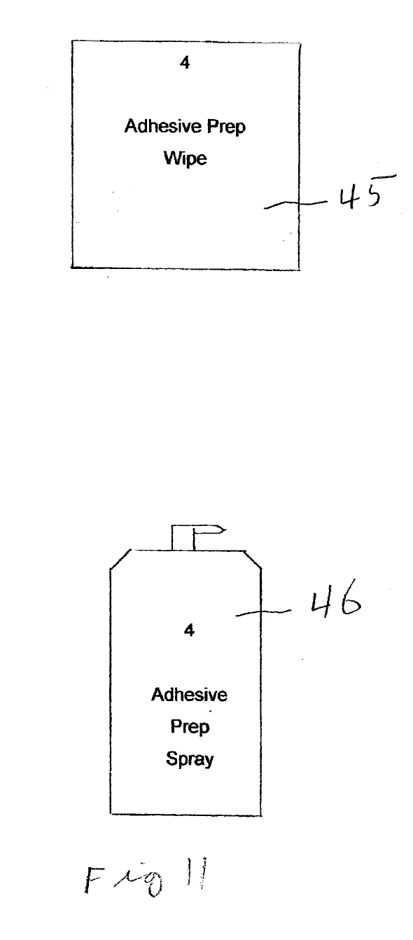

FIG. 11 shows alternative indicia that may be used in place of indicator 34, that is for an adhesive prep. Indicator 45 is for an adhesive prep wipe, and indicator 46 is for an adhesive prep spray.

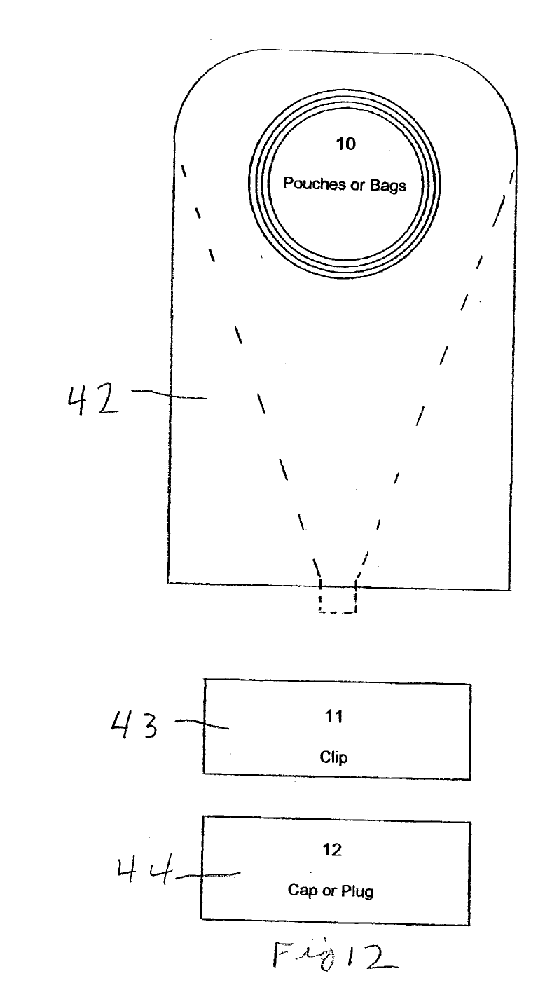

There are some patients who have both a colostomy or ileostomy and also a urostomy. To remind such patients to have both pouches available, along with closure means, the indicia shown in FIG. 12 may replace indicators 40 and 41. Indicator 42 provides a reminder that both types of pouch must be available. Indicator 43 is an indicator for a clip 22 to close pouch 20, and indicator 44 is an indicator for a cap or plug to close pouch 21.

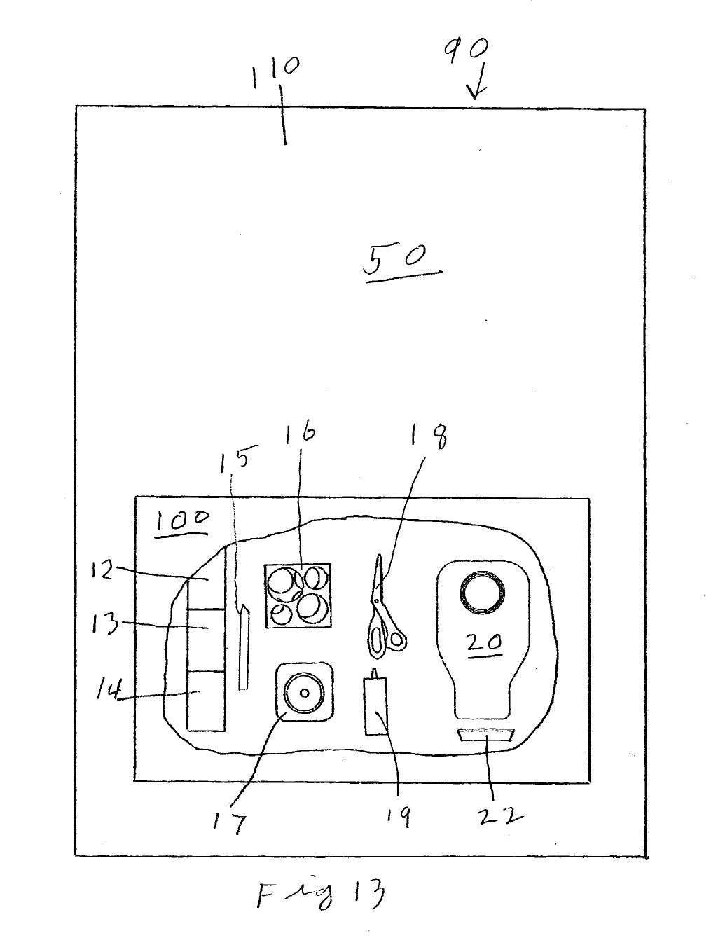

When a patient is first sent home from the hospital, after a colostomy, ileostomy, or urostomy, it is well for him or her to have an ostomy care system having all the supplies and implements needed, as well as a display arrangement as an aid to their use. FIG. 13 shows such an ostomy care system. Ostomy care system 90 includes a kit 100 containing skin preparation products including an adhesive remover 12, a moist wipe 13 and an adhesive prep 14. Ostomy care system 90 also includes a pencil 15, a measuring guide 16, a wafer 17, scissors 18, and paste 19, as well as pouch 20 and clip 22. A display arrangement, preferably display arrangement 50, is included in ostomy care system 90 to assist the patient or caregiver in providing the required ostomy care.

While the above description has presented various specific embodiments of the invention, it is not intended to be taken as limiting the invention. It is intended to provide teaching concerning the best modes known at the present time for practicing the invention. The scope of the invention is to be taken from the claims, which follow.

I claim:

1. A display arrangement for organizing and presenting supplies and implements needed for ostomy care, said display arrangement comprising:
at least one display surface for said supplies and implements, said display surface including alphamerically sequenced indicia disposed thereon, said alphamerically sequenced indicia including:
at least one indicator for at least one skin preparation product;
at least one indicator for stoma measurement;
at least one indicator for at least one wafer cutting implement;
at least one indicator for a wafer;
at least one indicator for at least one adhesive product; and
at least one indicator for at a pouch.

2. A display arrangement, according to claim 1, wherein said at least one display surface comprises a surface of at least one board or at least one tray.

3. A display arrangement, according to claim 1, wherein said at least one indicator for at least one skin preparation product includes an indicator for an adhesive remover.

4. A display arrangement, according to claim 3, wherein said indicator for an adhesive remover comprises an indicator for an adhesive remover wipe.

5. A display arrangement, according to claim 1, wherein said at least one indicator for at least one skin preparation product includes an indicator for a moist wipe.

6. A display arrangement, according to claim 1, wherein said at least one indicator for at least one skin preparation product includes at least one indicator for at least one adhesive prep product.

7. A display arrangement, according to claim 6, wherein said at least one indicator for at least one adhesive prep product includes at least one indicator for an adhesive prep wipe.

8. A display arrangement, according to claim 6, wherein said at least one indicator for at least one adhesive prep product includes at least one indicator for an adhesive prep spray.

9. A display arrangement, according to claim 1, wherein said at least one indicator for stoma measurement includes an indicator for a measurement guide having circle cutouts, said circle cutouts having disparate diameters.

10. A display arrangement, according to claim 1, wherein said at least one indicator for stoma measurement includes at least one indicator for a marking device for marking said wafer.

11. A display arrangement, according to claim 1, wherein said at least one indicator for wafer cutting includes at least one indicator for scissors.

12. A display arrangement, according to claim 1, further including an indicator for at least one of a clip, cap, or plug to close said pouch.

13. A display arrangement, according to claim 1, further comprising an indicator for at least one of a trash receptacle, or a biohazard receptacle.

14. A display arrangement, according to claim 1, wherein said display surface has at least one indentation or at least one elevated portion to prevent sliding of at least one implement or at least one supply.

15. A display arrangement, according to claim 1, wherein said display surface includes at least one paper or cardboard sheet having said indicia printed thereon.

16. A display arrangement, according to claim 1, comprising at least two display surfaces connected by at least one hinge whereby said display arrangement may be folded for storage or travel, said at least one hinge running from top to bottom.

17. A display arrangement, according to claim 1, comprising two display surfaces connected by a hinge whereby said display arrangement may be folded for storage or travel, said hinge running from side to side.

18. A display arrangement, according to claim 1, wherein said at least one indicator for a pouch includes at least one indicator for a plurality of types of pouch.

19. A display arrangement, according to claim 1, wherein said at least one display surface comprises surface(s) of at least one sheet of paper or cardboard having said indicia disposed thereon, said at least one sheet of paper or cardboard covered by a transparent layer.

20. An ostomy care system, said ostomy care system comprising:
at least one skin preparation product;
at least one device for stoma measurement;
at least one wafer cutting implement;
at least one wafer;
at least one adhesive product;
at least one pouch;
at least one display arrangement for said supplies and implements, said display arrangement including alphamerically sequenced indicia disposed thereon; said alphamerically sequenced indicia including:
at least one indicator for at least one skin preparation product;
at least one indicator for stoma measurement;
at least one indicator for at least one wafer cutting implement;
at least one indicator for a wafer;
at least one adhesive product; and
at least one indicator for a pouch.
